(19) United States
(12) Patent Application Publication
(10) Pub. No.: US 2006/0257385 A1
(43) Pub. Date: Nov. 16, 2006

(54) Dietary supplement formulations for improved delivery of coenzyme Q10 and methods of administration

(76) Inventor: Jarrow L. Rogovin, Los Angeles, CA (US)

Correspondence Address: MCCARTER & ENGLISH LLP;CITYPLACE I, 185 ASYLUM STREET, HARTFORD, CT, 06103, US

(21) Appl. No.: 11/404,519

(22) Filed: Apr. 14, 2006

Related U.S. Application Data
(60) Provisional application No. 60/671,404, filed on Apr. 14, 2005.

(51) Int. Cl. A61K 38/43; A61K 9/48; A61K 9/20; A61K 9/14; A61K 36/53

(52) U.S. Cl. 424/94.1; 424/451; 424/464; 424/745; 424/487

(57) ABSTRACT

A dietary supplement comprising Coenzyme Q10 (i.e. ubiquinone) and a bioadhesive substance is described. Derivatives of Coenzyme Q10 may also be used in the dietary supplement. The dietary supplement may be provided to an individual to increase blood plasma levels of Coenzyme Q10.

### REFERENCES RELATED TO THE APPLICATION

Under 35 U.S.C. § 119(e) this application claims the benefit of U.S. Provisional Application No. 60/671,404 filed Apr. 14, 2005, which is hereby incorporated by reference in its entirety.

### FIELD OF INVENTION

In one aspect, the present invention relates to a formulation comprising Coenzyme Q10, or derivatives of Coenzyme Q10, and a bioadhesive substance. The formulation can be provided to an individual in the form of a dietary supplement to increase the uptake of Coenzyme Q10 to the blood. The present invention also relates to methods of increasing the level of Coenzyme Q10 in the blood plasma of an individual by administration of a dietary supplement comprising Coenzyme Q10 and a bioadhesive substance.

### BACKGROUND OF THE INVENTION

Coenzyme Q10, also known as ubiquinone (herein referred to as “CoQ10”), supports heart function as a component of the electron transport system, and as an antioxidant protects mitochondrial membranes and cholesterol from oxidation. Because CoQ10 is of high molecular weight and is poorly soluble in water, various approaches have been taken to improve the dissolution and bioavailability of CoQ10 in dietary supplements. For example, CoQ10 has been complexed with cyclodextrins or solubilized with medium chain triglycerides to improve dissolution and availability of CoQ10.

Bioadhesives are generally defined as substances that adhere to a biological tissue for an extended period of time. Bioadhesives can be used to solve bioavailability problems by increasing the time that active ingredient resides at the site of absorption of the active ingredient. For example, mucoadhesants are a type of bioadhesive material that binds to the mucin layer of a biological membrane. Transmucosal delivery of biologically active substances offer advantages because mucus membranes are relatively permeable, allowing for the rapid uptake of an active substance into the systemic circulation and avoiding first pass metabolism. Mucoadhesants can also increase the residence time during which a substance may be absorbed, further increasing uptake.

Because CoQ10 has limited solubility in water, various formulations have been tried to improve the absorption of CoQ10 administered in dietary supplements. However, the previous attempts to improve absorption have been inadequate or have other drawbacks. There exists a need for a dietary supplement containing CoQ10 in a formulation that results in improved absorption of the CoQ10 by an individual.

### SUMMARY OF THE INVENTION

In one aspect, the present invention relates to a dietary supplement comprising CoQ10 and a bioadhesant. Derivatives of CoQ10, such as for example esterified CoQ10 or other CoQ10 complexes, may also be used in the dietary supplements of the present invention. The bioadhesant may be a synthetic polymer such as poly (acrylic acid), hydroxypropyl methylcellulose, or poly (methylacrylate) derivatives. Alternatively, the mucoadhesant may be a naturally occurring polymer such as hyaluronic acid. Preferably, the bioadhesive should be lipophilic. The dietary supplement may be provided in any appropriate form for administration to an individual, such as a capsule, tablet, powder, gel, liquid, bar, or in a single serving tube.

The dietary supplement may include additional ingredients, such as a lipid and an antioxidant for lipid stability. The dietary supplement may also contain excipients. When the dietary supplement is provided in a capsule or tablet, the dietary supplement may also include an enteric coating to protect the capsule or tablet as it passes through the stomach.

In another aspect, the present invention relates to methods for increasing the level of CoQ10 in an individual by administration of a dietary supplement comprising CoQ10, or a derivative of CoQ10, and a bioadhesant.

In yet another aspect, the present invention relates to methods for manufacturing a dietary supplement comprising CoQ10, or a derivative of CoQ10, and a lipid mucoadhesant.

### DETAILED DESCRIPTION OF PREFERRED EMBODIMENTS

In a first aspect, the present invention is directed to a dietary supplement comprising CoQ10 and a bioadhesant. The CoQ10 is preferably derived from fermentation and consists substantially of the natural trans configuration synthesized by humans, although the invention is not limited in this regard and any appropriate form or derivative of CoQ10 may be used. The dietary supplement contains sufficient CoQ10 to increase the level of CoQ10 in the blood plasma of an individual. In preferred embodiments, the dietary supplement contains between about 10 mg to about 1000 mg of CoQ10, preferably between 30 mg to about 300 mg of CoQ10, and more preferably between about 100 mg and about 200 mg of CoQ10.

CoQ10 derivatives comprising CoQ10 complexed or combined with other molecules, may be used in the dietary supplements of the present invention. Any appropriate form of CoQ10 that may be combined with a bioadhesant may be used in the dietary supplement. For example, esterified ubiquinones, such as for example acetyl CoQ10, succinyl CoQ10, or propionyl CoQ10, may be used. Alternatively, CoQ10 complexes, such as polyoxyethanyl-alpha-tocopheryl sebacate (PTS)-CoQ10 complex or CoQ10-cyclodextrin complex, may be used. In the following description of preferred embodiments, it should be understood that a CoQ10 derivative may be used in place of CoQ10 in the embodiments described.

In addition to CoQ10, the dietary supplement includes a bioadhesant. Preferably, the bioadhesant is lipophilic, although the invention is not limited in this regard. The bioadhesant may be a mucoadhesant. Any appropriate mucoadhesant known to those skilled in the art may be used in the composition. Examples of bioadhesants that may be used in the composition include synthetic polymers, such as poly (acrylic acid), hydroxypropyl methylcellulose, or poly (methyl-acrylate) derivatives. These materials may be copolymerized with polyethylene glycol (PEG) or poly (vinyl pyrrolidone) (PVP) to improve interpenetration and interdiffusion into the mucosal surface.

In one embodiment of the invention, polycarbophil is used as the bioadhesant, such as for example the polycarbophil manufactured by Noveon, Inc. of Cleveland, Ohio. Polycarbophils are polymers of acrylic acid crosslinked with divinyl glycol.

Naturally occurring polymers, such as, for example, hyaluronic acid, may also be used as bioadhesants in the formulations of the present invention.

The dietary supplements may contain nutrients in addition to CoQ10. For example, the dietary supplements may contain vitamins or other ingredients. The dietary supplements may also contain a lipid, such as for example phospholipids, a medium chain triglyceride, diglycerides or monoglycerides, and may also contain an antioxidant for lipid stability. Antioxidants that may be used for this purpose include, without limitation, gamma tocopherol, alpha lipoic acid, rosemary, ascorbyl palmitate or any other lipid antioxidant known to those skilled in the art. Additionally, excipients may be added to aid in tableting, as carriers, or for any other reason associated with the manufacture or storage of dietary supplements.

In one embodiment, the dietary supplement is provided in form of a softgel. The softgel may be made from any material known to those skilled in the art, such as for example gelatin, glycerin and water. The softgel may contain between 10 mg and 1000 mg, and preferably between about 30 mg and about 200 mg, of CoQ10 and between 10 mg and 1000 mg, and preferably between about 30 mg to about 500 mg, of a bioadhesant, such as for example polycarbophil. The softgel may further comprise between 10 mg and 1000 mg of a lipid, such as for example a medium chain triglyceride, diglycerides or monoglycerides. In addition, the softgel may contain an antioxidant for lipid stability. The softgel may also contain another lipid, such as phospholipids (for example, phosphatidylcholine) in a range of 100 mg to 1000 mg. Antioxidants that may be used for this purpose include, without limitation, gamma tocopherol, alpha lipoic acid, rosemary, ascorbyl palmitate or any other lipid antioxidant known to those skilled in the art.

Examples of embodiments of the invention are provided below. It will be understood that the following examples are provided as an aid to understanding the invention, and are not intended to limit the invention in any way.

Example 1 - Dietary Supplement in Capsule Form

| | |
|---|---|
| CoQ10 | Between 30 mg to 200 mg |
| Bioadhesant (e.g.Polycarbophil) | to capacity of capsule |
| Gamma tocopherol | 4 mg |
| Enteric coating | Optional |
| Excipients | Optional |

Example 2 - Dietary Supplement in Tablet Form

| | |
|---|---|
| CoQ10 | Between 10 mg to 100 mg |
| Bioadhesant (e.g.Polycarbophil) | Between 10 mg to 1200 mg |
| Gamma tocopherol | 4 mg |
| Enteric coating | Optional |
| Excipients | Optional |

Example 3 - Dietary Supplement in Softgel Form

| | |
|---|---|
| CoQ10 | Between 10 mg to 1000 mg |
| Bioadhesant (e.g. polycarbophil) | Between 10 mg to 1000 mg |
| Lipid | Between 10 mg to 1000 mg |
| Antioxidant | Between 4 mg to 200 mg |

In another aspect, the present invention provides methods for increasing the level of CoQ10 in the plasma of an individual by administration of a dietary supplement comprising CoQ10 and a bioadhesant. Preferably, the administration of the dietary supplement will result in a plasma concentration of CoQ10 of between about 2 μg/ml and about 4 μg/ml, as compared to typical plasma CoQ10 plasma levels of between 0.6 μg/ml to 0.8 μg/ml. The dietary supplement may be administered in the form of one or more tablets, capsules, or softgels, in the form of a liquid or gel, or in a bar or other nutritional supplement. The amount of dietary supplement to be administered depends on the quantity of CoQ10 in each dose (i.e. tablet, capsule, softgel, etc.), and the weight of the individual. The quantity to be administered to achieve a desired plasma level can be easily determined by a person skilled in the art.

The dietary supplements of the present invention can be manufactured using methods known to those skilled in the art. The components may be mixed and placed in the proper form for preparation of tablets, capsules, soft gels liquids or any other desired form using production known production methods.

As will be recognized by those of ordinary skill in the art based on the teachings herein, numerous changes may be made to the above-described embodiments of the invention without departing from its scope as described above and defined in the appended claims. For example, combinations of CoQ10 or one or more derivatives of CoQ10 may be used, and combinations of bioadhesants may be used in a dietary supplement formulation. Accordingly, this detailed description of preferred embodiments is to be taken in an illustrative, as opposed to a limiting, sense.

What is claimed:

1. A dietary supplement comprising Coenzyme Q10 and a bioadhesant.

2. The dietary supplement of claim 1, wherein the dietary supplement contains between about 10 mg and about 1000 mg of Coenzyme Q10.

3. The dietary supplement of claim 1, wherein the dietary supplement contains between about 30 mg and about 200 mg of Coenzyme Q10.

4. The dietary supplement of claim 2, wherein the dietary supplement contains between about 10 mg and about 1000 mg of a bioadhesant.

5. The dietary supplement of claim 3, wherein the dietary supplement contains between about 30 mg and about 500 mg of a bioadhesant.

6. The dietary supplement of claim 4, wherein the bioadhesant is selected from the group consisting of poly (acrylic acid), hydroxypropyl methylcellulose, poly (methyl-acrylate) derivatives, copolymers of poly(acrylic acid) and polyethylene glycol (PEG), copolymers of poly(acrylic acid) and poly (vinyl pyrrolidone) (PVP), and combinations thereof.

7. The dietary supplement of claim 5, wherein the bioadhesant is polyacrylic acid.

8. The dietary supplement of claim 5, wherein the bioadhesant is hyaluronic acid.

9. The diestary supplement of claim 5, wherein the bioadhesant is polycarbophil.

10. The dietary supplement of claim 5, further comprising a combination of at least one lipid and an antioxidant for lipid stability.

11. The dietary supplement of claim 10, wherein the lipid is selected from the group consisting of phospholipids, medium chain triglycerides, diglyceride, monoglycerides, and combinations thereof.

12. The dietary supplement of claim 11, wherein the antioxidant for lipid stability is selected from the group consisting of gamma tocopherol, alpha lipoic acid, rosemary, ascorbyl palmitate, and combinations thereof.

13. The dietary supplement of claim 4, wherein the dietary supplement is in the form of a tablet, capsule, softgel, liquid, powder or nutritional bar.

14. The dietary supplement of claim 5, wherein the dietary supplement is in the form of a tablet, capsule, softgel, liquid, powder or nutritional bar.

15. A dietary supplement comprising a derivative of CoQ10 and a bioadhesant.

16. The dietary supplement of claim 15, wherein the derivative of CoQ10 is selected from the group consisting of acetyl CoQ10, succinyl CoQ10, propionyl CoQ10, polyoxyethanyl-alpha-tocopheryl sebacate (PTS)-CoQ10 complex, CoQ10-cyclodextrin complex, and combinations thereof, and the bioadhesant is selected from the group consisting of poly (acrylic acid), hydroxypropyl methylcellulose, poly (methyl-acrylate) derivatives, copolymers of poly(acrylic acid) and polyethylene glycol (PEG), copolymers of poly(acrylic acid) and poly (vinyl pyrrolidone) (PVP), and combinations thereof.

17. A method for increasing the level of Coenzyme Q10 in an individual comprising the step of administering to the individual a dietary supplement comprising Coenzyme Q10 and a bioadhesant.

18. The method of claim 14, wherein the dietary supplement is administered to achieve a blood plasma concentration of Coenzyme Q10 of between about 2 μg/ml and about 4 μg/ml.

19. The method of claim 14, wherein the dietary supplement contains between about 10 mg and about 1000 mg of Coenzyme Q10.

20. The method of claim 14, wherein the dietary supplement contains between about 30 mg and about 200 mg of Coenzyme Q10.
